(19) United States
(12) Patent Application Publication
(10) Pub. No.: US 2021/0322698 A1
(43) Pub. Date: Oct. 21, 2021

(54) Airway Pressure Release Ventilator

(71) Applicant: Orics Industries, Inc., Farmingdale, NY (US)

(72) Inventors: Ori Cohen, Albertson, NY (US); Isaac Cohen, Oakland, CA (US); Carlos Milla, Redwood City, CA (US)

(73) Assignee: Orics Industries, Inc., Farmingdale, NY (US)

(21) Appl. No.: 17/234,308

(22) Filed: Apr. 19, 2021

Related U.S. Application Data
(60) Provisional application No. 63/012,192, filed on Apr. 19, 2020.

(51) Int. Cl. A61M 16/00; A61M 16/12

(52) U.S. Cl. CPC A61M 16/024; A61M 16/12; A61M 2202/0208; A61M 2016/003; A61M 2016/0027

(57) ABSTRACT

A system and method for ventilating includes a holding tank, such as, for example, a medical sealed apparatus, for storing of gases. The holding tank stores at least pressured oxygen, for example O₂, though the holding tank may store a pressurized blend of oxygen and air. This blend may include a 50-50 mix of oxygen and air. The pressure applied to the gases in the holding tank may include a range from 5 pounds force per square inch to 20 pounds force per square inch. The blend of oxygen and air, for example, under pressure in the holding tank, the system and method for ventilating of the present disclosure no longer requires an electronic mechanical system, such as, for example, a pump or motor on the inhalation control and the exhalation control to the ventilating system and method.

### BACKGROUND

### Field of the Disclosure

The present disclosure generally relates to medical equipment generally, and more particularly, to a ventilator.

### Description of Related Art

This section intends to provide a background discussion for a clear understanding of the disclosure herein but makes no claim nor any implication as to what is the relevant art for this disclosure.

Various medical equipment are currently employed in supporting impaired human breathing, most commonly referred to a ventilators. A ventilator may be defined as a device machine that provides mechanical ventilation by moving breathable air into and out of the lungs, thereby delivering breaths to a patient who is impaired or physically unable to breathe or breath sufficiently. While numerous ventilator designs exist, the most advanced systems rely on computer controlled systems though a simple, hand-operated bag valve mask design are still in use.

With the recent rise of The Coronavirus or Covid 19, the shortage of ventilators has become more glaringly more apparent to public health and safety experts as well as the general public. The most severely impaired Covid 19 patients require ventilators once admitted into intensive care units in hospitals. Naturally, ventilators may also be found in-home care, mobile emergency medicine and as part of anesthesia machines.

Ventilators are currently implemented using an electro-mechanical system to push air through the trachea and into a patient's lungs. These systems rely on motors or pumps to effectively allow a patient to breath mechanically. Electro-mechanical systems like motors and pumps have a predictable fail rate time, require maintenance, consume high amounts of energy, generate heat waste and add bulk to a ventilator design. Consequently, many ventilation machines use electric motors and brushless driven turbine to control the pressurized air flow during both inhalation and exhalation of the lungs, without depending on pressurized gas supply.

### SUMMARY

The present disclosure includes a system and method for ventilating that eliminates the need for a motorized system to push air into a patient's lungs.

In one embodiment of the disclosure, a system for ventilating includes one or more holding tanks, such as a medical sealed apparatus, for storing of gases. The holding tank stores at least pressured oxygen, for example O₂, though the holding tank may store a pressurized blend of oxygen and air. This blend can be in concentration of 100% air to 100% oxygen. This blend may include a 21%-100% oxygen concentration. The pressure applied to the gases in the holding tank may include a range from 5 pounds force per square inch to 20 pounds force per square inch. The blend of oxygen and air, for example, under pressure in the holding tank, the system and method for ventilating of the present disclosure no longer requires a motorized mechanical system, such as, for example, a pump or turbine on the inhalation control and the exhalation control to the ventilating system and method.

In another embodiment of the disclosure, the system for ventilating a patient includes a gas holding reservoir tank, such as, for example, a medical sealed apparatus, for storing of pressurized gases, including, for example, oxygen (O₂) and air. Coupled with the medical sealed apparatus is a breathing tube that is to be fit with the patient. The system for ventilating also includes a controller, also coupled with the breathing tube, for blending gasses such as oxygen and air into a mix that may be set, for example, by a medical professional. This controller may be integrated as part of the gas holding reservoir tank. The controller also managing one or more inhalation cycles of the mixed pressurized gasses into the breathing tube. The controller also manages one or more exhalation cycles from the breathing tube each of which generates an output.

In another embodiment, the system's control mechanism may be altered for the mix of the pressurized gases in response to the output from the one or more exhalation cycles.

In yet another embodiment, the mix of the pressurized gases comprises 50 parts oxygen and 50 parts air.

In still another embodiment, the mix of pressurized gases comprises 5 pounds force per square inch to 20 pounds force per square inch.

In yet still another embodiment, the control mechanism includes a computer for managing an onset of the inhalation cycle in response to a volume of the output from the exhalation cycle, and a timer for measuring one or more purge time between one or more pairs of inhalation and exhalation cycles.

In yet another embodiment, the control mechanism further includes a device for controlling a pressure of the one or more exhalation cycles and for allowing for carbon dioxide release.

In yet another embodiment, the control mechanism further comprises one or more closed-loop sensors for sensing pressure and flow of the mix. Here, the closed-loop sensor(s) may control one or more inhalation cycles and the one or more one exhalation cycles.

In yet another embodiment, the system further includes a monitor for monitoring the output to provide a constant and uniform purge of oxygen and air and for minimizing supply pressure fluctuation.

In yet another embodiment, the system further includes a force gas flow device for generating a gas flow of one or more pressure gradients.

In another embodiment of the present disclosure, a system for medical ventilating patients includes one or more medical sealed apparatus for storing pressurized gases, where the pressurized gases include at least of oxygen and air. The system further includes one or more breathing tube, as well as a control mechanism coupling the one or more breathing tubes with the one or more medical sealed apparatus for storing pressurized gases. The control mechanism blends the pressured oxygen and the pressurized air into a mix, while managing one or more inhalation cycles of the mixed pressurized gases into the breathing tube and to the patient(s). The control mechanism also manages one or more exhalation cycles through the breathing tube such that each exhalation cycle may generate an output. The control mechanism also includes a force gas flow device for generating a gas flow of one or more pressure gradients as well as a monitor for monitoring the pressure gradient before each inhalation and each exhalation cycle.

In another embodiment, the control mechanism may be altered so the mix of the pressurized gases in response to the output from the at least one exhalation cycle.

In yet another embodiment, the mix of at least one pressurized gases includes a controlled mix of air and oxygen from 0% oxygen to 100% oxygen.

In still yet another embodiment, the mix of pressurized gases comprises 5 pounds force per square inch to 20 pounds force per square inch.

In still yet another embodiment, the control mechanism includes a computer for managing an onset of each inhalation cycle in response to a volume of the output from the exhalation cycle, as well as a timer for measuring one or more purge times between each pair of inhalation and exhalation cycles.

In yet another embodiment, the control mechanism further controls a pressure of the at least one exhalation cycle to allow for carbon dioxide release.

In yet another embodiment, the monitor further includes a monitoring controller for providing a constant uniform purge of oxygen and air, and for minimizing supply pressure fluctuation.

In yet another embodiment, the system also includes one or more closed-loop sensors for sensing pressure and flow of the mix of pressured gasses such that the sensor(s) controls the one or more inhalation and exhalation cycles.

In yet another embodiment of the disclosure, a method for ventilating medical patients includes storing pressurized gases, such as, for example, oxygen and pressurized air, in one or more medical sealed apparatus. The method includes coupling the medical sealed apparatus with one or more breathing tubes, and blending the pressurized gases to a mix. Thereafter, the method includes managing at least one inhalation cycle of the mix of pressurized gases into the breathing tube, as well as managing at least one exhalation cycle of through the one breathing tube. The method then generates an output from the exhalation cycle, and generates a gas flow of at least one pressure gradient during the exhalation cycle. The method also includes the step of monitoring the pressure gradient before the output is generated.

In another embodiment, the method further includes the step of altering the mix of the pressurized oxygen and the pressurized air in response to the output during the at least one exhalation cycle.

In yet another embodiment, the mix of pressurized gases includes 50 parts oxygen and 50 parts air.

In still yet another embodiment, the mix of pressurized gases comprises 5 pounds force per square inch to 20 pounds force per square inch.

In still yet another embodiment, the method also includes managing an onset of the inhalation cycle in response to a volume of the output from the exhalation cycle, as well as measuring at least one purge time between at least one pair inhalation and exhalation cycles.

In still yet another embodiment, the method also includes controlling exhalation cycle pressure to allow for carbon dioxide release.

In another embodiment, the method further includes providing a constant and uniform purge of oxygen and air while minimizing supply pressure fluctuation.

In another embodiment, the method also includes the step of closed-loop sensing of pressure and flow of the mix to control one or more inhalation and exhalation cycles.

### BRIEF DESCRIPTION OF THE DRAWINGS

The present disclosure and its various features and advantages can be understood by referring to the accompanying drawings by those skilled in the art relevant to this disclosure. Reference numerals and/or symbols are used in the drawings. The use of the same reference in different drawings indicates similar or identical components, devices or systems. Various other aspects of this disclosure, its benefits and advantages may be better understood from the present disclosure herein and the accompanying drawings described as follows:

FIG. 1 illustrates an embodiment of the present disclosure;

FIG. 2 illustrates yet another embodiment of the present disclosure; and

FIGS. 3-7 illustrate additional embodiments of the present disclosure.

### DETAILED DESCRIPTION

The present disclosure includes a system and method for ventilating that does eliminates the need for a motorized system to push air into a patient's lungs.

In one aspect of the disclosure, a system for ventilating is detailed including one or more holding tanks, such as a medical sealed apparatus, for storing of gases. The holding tank stores at least pressured oxygen, for example O₂, though the holding tank may store a pressurized blend of oxygen and air. This blend may include a controlled mix of air and oxygen from 0% oxygen to 100% oxygen and air. The pressure applied to the gases in the holding tank may include a range from 5 pounds force per square inch to 20 pounds force per square inch. The blend of oxygen and air, for example, under pressure in the holding tank, the system and method for ventilating of the present disclosure no longer requires a motorized system, such as, for example, a pump or turbine on the inhalation control and the exhalation control to the ventilating system and method.

In another aspect of the disclosure, a method for ventilating medical patients includes storing pressurized gases, such as, for example, oxygen and pressurized air, in one or more medical sealed apparatus. The method includes coupling the medical sealed apparatus with one or more breathing tubes, and blending the pressurized gases to a mix. Thereafter, the method includes managing at least one inhalation cycle of the mix of pressurized gases into the breathing tube, as well as managing at least one exhalation cycle of through the one breathing tube. The method then generates an output from the exhalation cycle, and generates a gas flow of at least one pressure gradient during the exhalation cycle. The method also includes the step of monitoring the pressure gradient before the output is generated.

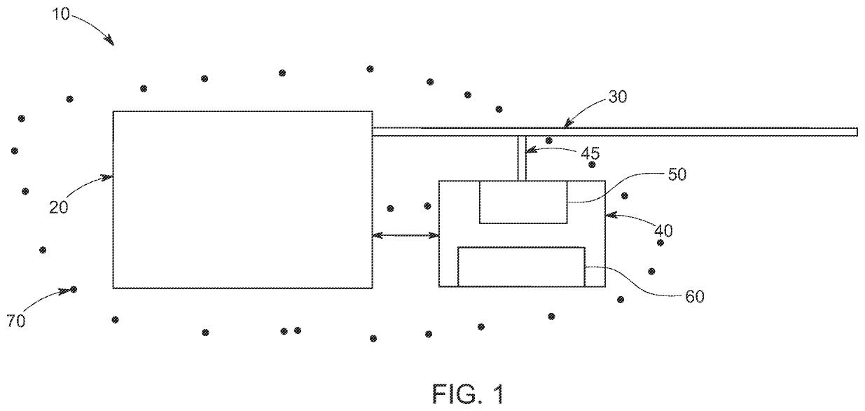

Referring to FIG. 1, a first embodiment of the present disclosure is illustrated. Here, a system 10 is depicted for ventilating patients in needs of supplemental respiration support. System 10 includes a medical sealed apparatus 20 for storing pressurized gases. Apparatus 20 can be realized by various means apparent to skilled artisans upon reading the teachings of disclosure herein. In one embodiment, apparatus 20 is a gas holding reservoir tank. In this regard, apparatus 20 is a storage unit for pressurized gases. In one embodiment, these pressurized gases include oxygen (O₂) and air. It should be apparent to skilled artisans upon reading the disclosure herein that other gases may be included for other applications including, for example, anesthesia.

Ventilating system 10 further includes breathing tube 30. Breathing tube 30 is mechanically coupled to gas holding reservoir tank 20. Breathing tube 30 ultimately delivers the pressurized gas to the patient in need of supplemental respiration support. It should be noted that several breathing tubes (not shown) may be mechanically coupled to gas holding reservoir tank 20 to support various applications including servicing several patients in simultaneous need of supplemental respiration support.

Moreover, ventilating system 10 also includes a control mechanism 40. Control mechanism 40 performs a number of functions. To realize this aim, control mechanism 40 is mechanically coupled with gas holding reservoir tank 20 and breathing tube 30. One function performed by control mechanism 40 is for managing the blending percentages of the pressurized gases in holding reservoir tank 20. A medical professional, after examining a patient in need of supplemental respiration support may conclude that a particular mix of oxygen (O₂) and air is required given the state of the patient's lungs. Typically, the mix of the blended pressurized gasses is a controlled mix of air and oxygen from 0% oxygen to 100% oxygen though other recipes will be apparent to skilled artisans upon reviewing the disclosure herein. In one embodiment, the mix of pressurized gases delivered to the medical patient involved a range of 5 pounds force per square inch to 20 pounds force per square inch. Once the blended recipe is selected using control mechanism 40, gas holding reservoir tank 20 may enable the pressurized gas mix to be delivered down through the breathing tube 30 to the medical patient.

Another function performed by control mechanism 40 is in the overall management of ventilator system 10. More particularly, control mechanism 40 manages the inhalation cycles in terms of delivery of the mixed pressurized gasses through the breathing tube 30 to the medical patient. The inhalation cycles are set by a medical professional and inputs to this include the damage to the medical patient's pulmonary system, the level of consciousness, as well as the volume of gas exhaled back from the medical patient into breathing tube 30. Similarly, control mechanism 40 also manages the exhalation cycle of the medical patient by tracking the volume of gas flow back from the patient. This gas flow back is, for the purposes of the present disclosure, referred to as output.

It should be noted that control mechanism 40 may be realized by a computing system (not shown). By such an implementation, control mechanism 40 may also alter the mix of the pressurized gases from gas holding reservoir tank 20 through the breathing tube 30 to the medical patient in response to the patient's exhalation cycles. For example, if the patient's output increasingly contains carbon dioxide, reflective of an improving condition, control mechanism 40 may sense the output and automatically reduce the mix of oxygen (O₂) and air back through the breathing tube 30 or, in the alternative, alert the medical professional that a different mix is warranted.

Another feature of control mechanism 40 is that it may include a timer (not shown). The timer's function of control mechanism 40 is for measuring at least one purge time between each cycle of inhalation and the exhalation. This information may provide the medical professional insights on the patient's pulmonary status and whether a different mix of pressurized gases are needed.

Still another feature of control mechanism 40 is in the management of the exhalation function. In one embodiment, control mechanism 40 may include a device for monitoring and controlling the pressure of the patient's exhalation cycle, while allowing for the release of carbon dioxide. Given the design of system 10, control mechanism 40 can apply pressurized force of the gas mix delivered through breathing tube 30 to increase the intake of the mix into the patient's pulmonary system.

Control mechanism 40 may further include one or more closed-loop sensor(s) 50. Sensor 50, coupled to breathing tube 30 through sensing tube 45, serves the purpose of sensing the pressure and flow of the mix of gasses delivered to the medical patient. As a consequence, closed-loop sensor 50 may control the inhalation and exhalation cycles of system 10 as desired.

Moreover, control mechanism 40 may also include a force gas flow device (not shown). The purpose of the force gas flow device is for generating a gas flow of at least one pressure gradient from gas holding reservoir tank 20 to breathing tube 30 by means on control mechanism 40.

Control mechanism 40 may further include a monitor 60. Monitor 60 acts as device for monitoring the output from the patient during an exhalation cycle. Monitor 60 also may provide a constant and uniform purge of oxygen and air to minimize supply pressure fluctuation.

It should be noted that gas holding reservoir tank 20 and control mechanism 40 may be realized in single, integrated unit 70. In this arrangement, integrated unit 70 simple couples with breathing tube 30 in a direct fashion.

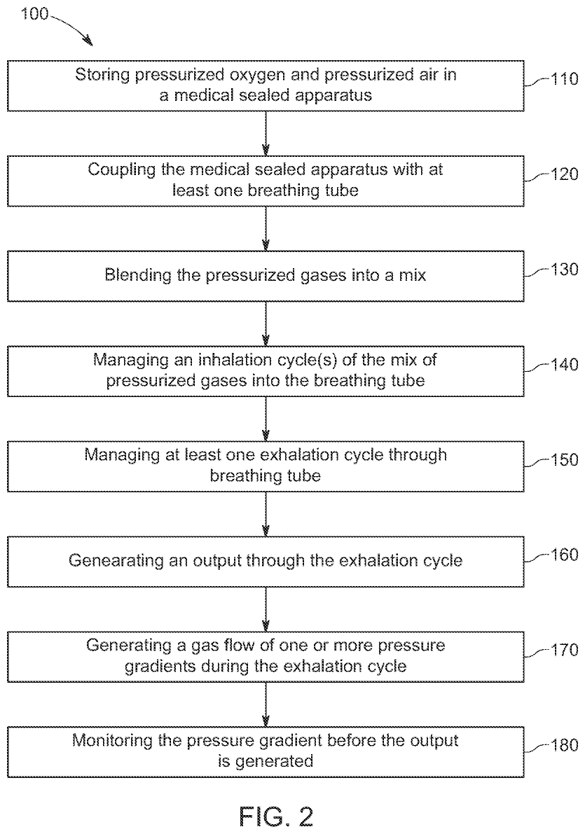

Referring to FIG. 2, a second embodiment of the present disclosure is illustrated. Here, a flow chart is depicted for a method 100 for ventilating medical patients. Method 100 of this embodiment begins with the step 110 of storing one or more pressurized gases in a medically sealed apparatus or, a gas holding reservoir tank. The pressurized gases stored in this step will include oxygen (O₂) and air. It should be apparent to skilled artisans upon reading the disclosure herein that other gases may be included for other applications including, for example, anesthesia.

Once the pressurized gases are stored in a medically sealed apparatus of step 110, the apparatus is then coupled 120 with one or more breathing tubes. This coupling, ultimately, is allow a specific mix of pressurized gas to flow through the breathing tube and enable the patient to ventilate their pulmonary system.

After the medically sealed apparatus is coupled with the breathing tube, method 100 then calls for blending 130 the pressurized gases into a desires mix to properly treat the patient. This mix is selected by the medical professional based on observation of the patient. In one embodiment, the mix is a controlled mix of air and oxygen from 0% oxygen to 100% oxygen. The resultant mix of gasses in the tank may include a pressurized range from 5 pounds force per square inch to 20 pounds force per square inch.

Once the pressurized blended, method 100 then calls for the step of managing 140 of one or more inhalation cycles of the mixed of pressurized gases into the breathing tube. Step 140 is followed by the step of managing 150 one or more exhalation cycle of through the at least one breathing tube,

As a consequence of steps 140 and 150, method 100 then generates 160 an output, as defined herein, through one or more of the exhalation cycles. This allows for the subsequent step 170 of generating a gas flow of one or more pressure gradients during the exhalation cycle. Finally, method 100 calls for the step 180 of monitoring the pressure gradient before the output is generated.

It should be noted that method 100 may also include additional steps in alternative embodiments. For example, method 100 may include the step (not shown) of managing an onset of the inhalation cycle in response to a volume of the output from the exhalation cycle. In so doing, method 100 may also include the step (not shown) of measuring at least one purge time between at least one cycle of inhalation and exhalation. Further, method 100 may also include the step (not shown) of controlling the pressure of at least one exhalation cycle to allow for carbon dioxide release from the medical patient.

Other embodiments to method 100 include the step (not shown) of providing a constant and uniform purge of oxygen and air. This step may be realized while minimizing supply pressure fluctuation. Additionally, method 100 may also include closed-loop sensing (not shown) of pressure and flow of the mix of the pressurized gasses. This is intended to control one or more cycles of inhalation and exhalation.

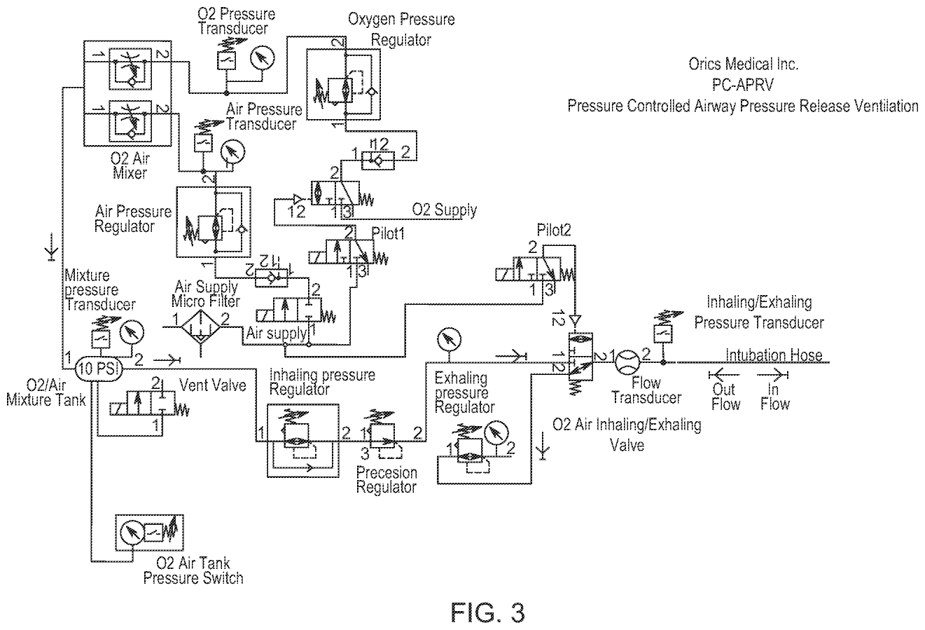

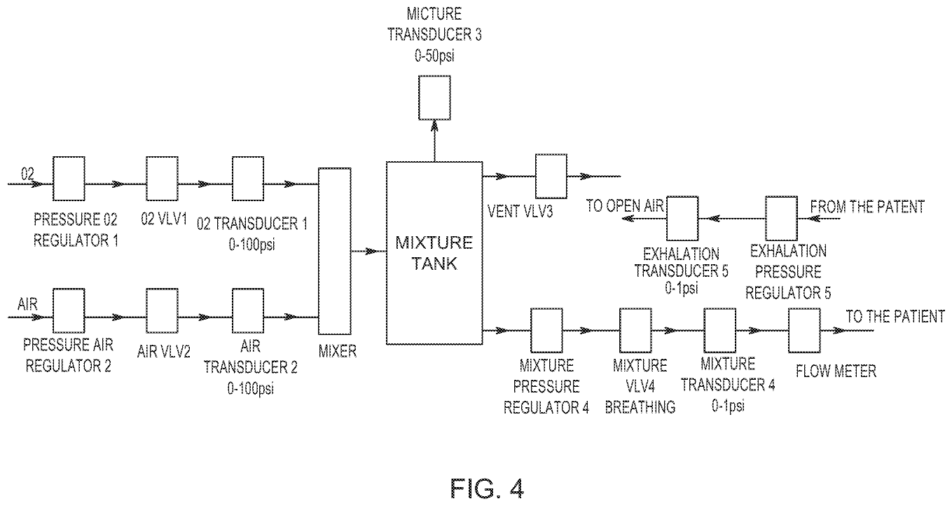

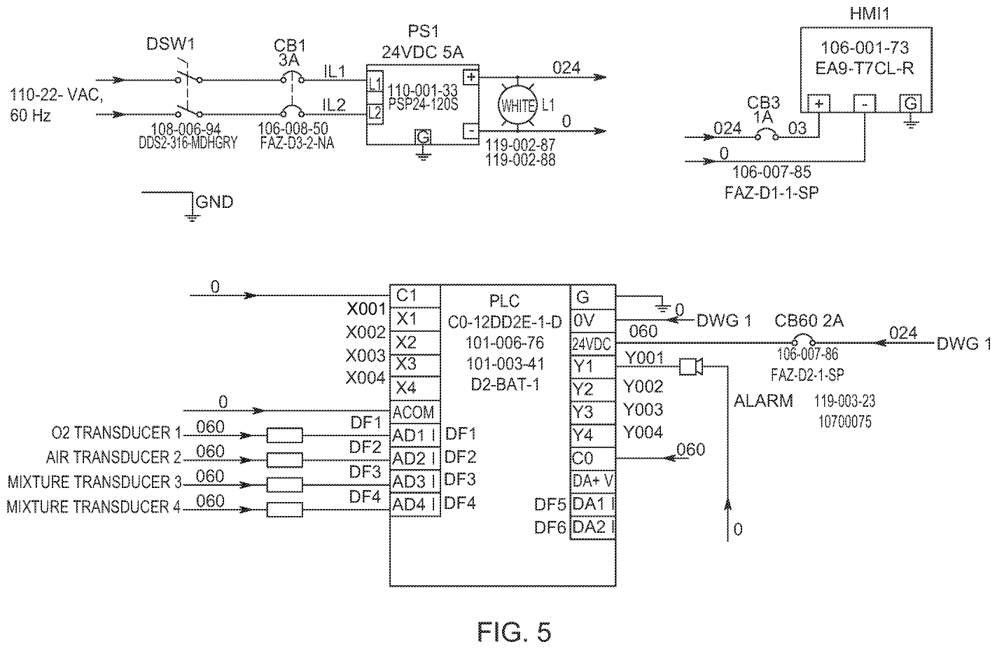

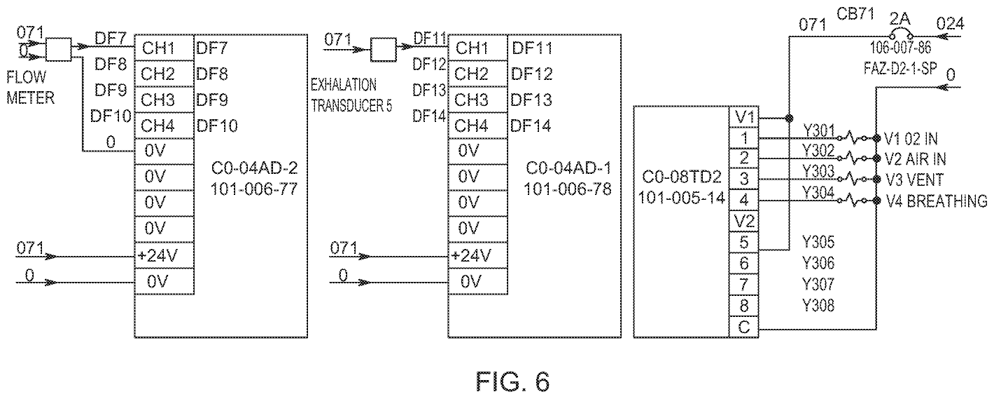

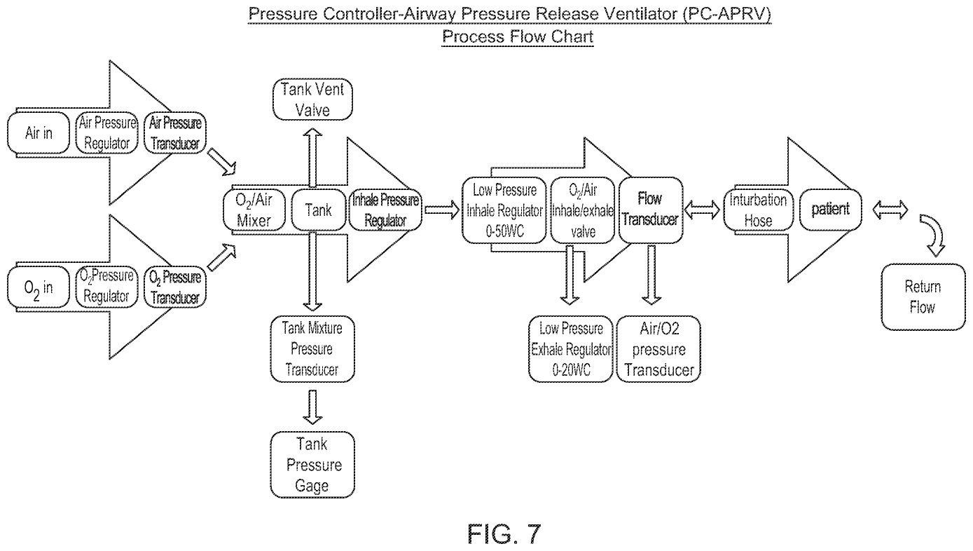

Referring to FIGS. 3-7, another set of embodiments of the present disclosure are illustrated. Here, the controlled pressure of air and oxygen (e.g., O₂), from an external supply, such as hospital central gas supply or free-standing supply tanks or depending on supply gasses quality and cleanliness, flow independently through user supplied filters. This flows from a line connected to a pressure regulator for air and another for oxygen (O₂), then through a valve for each pressure regulator, followed by a pressure transducer and, finally low flow, mixer for air and O₂ at the designated ports. One exemplary low flow, mixer is the Sechrist Model 3500

From a process flow, in another embodiment of the present disclosure, the enters with a ½″ port solenoid valve. In contrast, the oxygen (O₂), for safety purposes, enters via a pilot valve without an electric signal for operating the valve, but an air pilot signal from the ½″ port solenoid valve. The air and gas are pressure regulated to ensure the mixer receives constant flow regardless of potential supply fluctuations. Both inlet air and oxygen (O₂), are also pressured controlled using a pressure transducer;

Operationally, in an embodiment of the present disclosure, the medical professional adjusts the air to oxygen (O₂) ratio of the mixture with a flow control dial button provided on, for example, the Secrist Model 3500 mixer. The ratio can be from 21% oxygen (O₂), ambient air, to enriched oxygen (O₂) air mixture, up to 100% oxygen (O₂). The mixture then enters a stainless-steel holding reservoir tank. The volume of air and oxygen (O₂) in the tank is pressure controlled with a regulator and a pressure transducer. Once it reaches its desired pressure, it stops the supply of both air and oxygen (O₂).

The present disclosure may provide mechanical invasive, anesthesia free, lung ventilation for acute lung injury (ALI) or acute respiratory distress syndrome (ARDS) by releasing controlled pressure of air and oxygen mixed gas for inhalation and supporting controlled pressure exhalation, through an endotracheal tube (ETT) or other suitable patient interface.

It should be noted that the tank, according to an embodiment of the present disclosure, may have at least two ports for output. The first port is for venting the tank when the cycles are all done or if/when the system is stopped, while the second port is a controlled valve for the breathing apparatus.

According to another embodiment of the present disclosure the control valve may have three ports. The first port is for inflow, connecting the holding reservoir tank to the valve, while the second port is for outflow, connecting supply line of air/oxygen mix to the patient lungs connected to the breathing tube or Endotracheal Tube Medical (“ETT”). The third control valve port is for exhaust, connecting the patient's exhaled gasses (exhaust). This exhaust may be fed to various sources including, for example, a hospital supplied exhaust respiratory filter. It is contemplated that if patient's airways are infected, the exhaust respiratory filter will be heavily recommended. The outflow and exhaust ports hereinabove are controlled separately with pressure regulators and flow control meters.

According to another embodiment of the present disclosure, the ventilator system may be electrically coupled with a conventional wall plug socket, such as 110V or 220V, for example. The ventilator system may include an on/off switch to power on and off the system. The ventilator system valves, transducers, pressure regulators and flow meters may be wired, in one embodiment, to a programmable logic controller (PLC) with a touch screen human-machine-interface (HMI) display, for example. The control parameters may have a display representation to set operational parameters on the HMI screen. Here, operational parameters may be entered manually by the ventilator operator to conform to technical standards agreed upon in the medical and regulatory community. The touch screen HMI may also have a start/stop display button to initiate and terminate the ventilator system's operation. The touch screen display may have a graphic display of the closed-loop readouts from the controllers. In one embodiment, the programmable logic controller may be connected with a cellular SIM card/system, a wireless WiFi card, an ethernet port, or the like to enable remote control via an internet connection. As a consequence, the programmable logic controller may also have a USB port to enable coupling with USB devices though other standardized connectors and systems are contemplated by the disclosure herein.

In another embodiment, the ventilator system includes a gas supply to gas mixer. The gas supply to gas mixer includes various components cooperating together including oxygen pressure regulator, an oxygen pilot valve, a pressure transducer, an air pressure regulator, an air ½″ solenoid valve and a pressure transducer. Further, the ventilator system also includes fittings piping, as well as a gas mixer, such as, for example, Sechrist Model 3500hl Air Oxygen Mixer. Additionally, the ventilator system may include a mixed o2/air holding reservoir tank. This tank may be designed from stainless steel material and include a tank mixture transducer, a vent valve, various fitting and piping. The ventilator system here may further include a patient outflow port from tank, which may include a mixture pressure regulator, a mixture transducer, a flow meter, a three-way valve, an exhalation pressure regulator, as well as fittings and piping. Further, the ventilator system may also include various additional components including electrical switches, a programmable logic controller, a display, a wireless (WiFi or cellular for example) card, a USB port, and an Ethernet port.

In another embodiment of the present disclosure, a mechanical process flow narrative can be detailed as follows:
1. O₂ supply to Sechrist 3500 gas mixer
   a. O₂ supply connected to O₂ pressure regulator. The pressure regulator controls the O₂ gas pressure from the source into the ventilator
   b. Pressure regulator connected to Pilot valve. The Pilot valve controls the on and off flow based on reservoir holding tank demand via a pressure signal from the solenoid valve
   c. Pilot valve connected to pressure transducer. The pressure transducer translates the analog pressure into an electrical signal for digital control
   d. Pressure transducer connected to O₂ port on the Sechrist 3500. Connecting the O₂ pressurized, signal-transformed supply to the air/O₂ mixer
2. Air supply to Sechrist 3500 gas mixer.
   a. Air supply connected to Air pressure regulator. The pressure regulator controls the Air gas pressure from the source into the ventilator
   b. Pressure regulator connected to ½″ solenoid valve. The Pilot valve controls the on and off flow based on reservoir holding tank demand via an electrical signal from the PLC
   c. ½″ solenoid valve connected to pressure transducer. The pressure transducer translates the analog pressure into an electrical signal for digital control
   d. Pressure transducer connected to air port on the Sechrist 3500. Connecting the Air pressurized, signal-transformed supply to the air/O₂ mixer
3. Sechrist 3500 gas mixer has a mechanical circular dial controlling the ratio of O₂ to Air allowing O₂ concentration in the mixture from 21%-100%
4. Sechrist 3500 gas mixer to Reservoir Holding Tank provides the required Air/O₂ mixture to the reservoir holding tank
5. Reservoir Holding Tank to vent valve. The vent valve empties the reservoir holding tank upon termination of ventilation cycle or as safety control in the event a cycle needs to be aborted
6. Reservoir Holding Tank to Mixture pressure transducer. The pressure transducer translates the analog pressure in the reservoir holding tank into an electrical signal for digital control
7. Reservoir Holding Tank to ETT
   a. Reservoir holding tank to mixture inflow (inspiration, inhalation) pressure regulator. The pressure regulator controls the inflow pressure of mixed air/O₂. The inflow pressure parameters are controlled by the operator on the HMI display
   b. Pressure regulator to 3-way valve. During inflow cycle the ports from reservoir holding tank and the flow port open. The exhaust port is closed.
   c. 3-way valve to mixture pressure transducer. The mixture pressure transducer translates the analog pressure in the pressure regulator into an electrical signal for digital control
   d. Mixture transducer to bi-directional flow meter. The flow meter measures the rate of flow of the air/O₂ mixture inflowing to the ETT
   e. Flow meter to outflow (expiration, exhalation) pressure regulator. The outflow pressure regulator is set at the same pressure as the inflow pressure regulator during inflow of air/O₂ mixture into ETT. The outflow pressure regulator is set to a required outflow set pressure to prevent atelectasis
   f. Outflow pressure regulator to bi-directional flow meter. The flow meter measures the rate of flow of the outflowing (exhaled) gasses from the ETT and reports the Flow at Peak and End of exhalation.
   g. Flow meter to mixture pressure transducer. The mixture pressure transducer translates the analog pressure from the outflow from the ETT into an electrical signal for digital control
   h. Mixture transducer to 3-way valve. During outflow cycle the flow port and the exhaust port are open. The reservoir holding tank port is closed.
   i. 3-way valve to air filter. The air filter is optional. If the patient is infected the air filter is mandatory

In another embodiment of the present disclosure, the programming logic controller and HMI display may be configured as follows:
1. Wiring Logic
2. Control Parameters
   a. Pressure
      i. Pressure is controlled between the air and O₂ source and the air/O₂ mixer: PSI (Increments of 0.1 PSI; Min: 40 Max: 50)
      ii. Pressure is controlled inside the reservoir holding tank: PSI (Increments of 0.1 PSI; Min: 7 Max: 10)
      iii. Pressure is controlled from the reservoir holding tank to the ETT: 0 cm H₂O— 90 cm H₂O (increments of 1 cm H₂O; Alarms at <10 cm H₂O and >40 cm H₂O)
      iv. Pressure is controlled from ETT to exhaust port: 0 cm H₂O— 10 cm H₂O (increments of 1 cm H2O; Alarm at >10 cm H2O.
   b. Time
      i. Time is controlled for the inflow cycle 0.1-30 seconds (increments of 0.1 seconds; decimal; Alarm is set at >30 second1)
      ii. Time is controlled for the outflow cycle: 0.05-30 seconds (increments of 0.05 seconds; decimal; Alarm is set at <0.5 second and >30 seconds).
   c. Flow
      End Expiratory Flow to Peak Expiratory Flow alarm limits set at <0.50 and >0.80
3. Display Logic
   a. Start/Stop switch. Controls the start and stop of ventilation.
      i. Start alarms are set whenever a parameter is not inserted by the operator or,
      ii. O₂ and/or air supply pressure is >40 PSI iii. Holding reservoir pressure is >7 PSI
      iv. No signal is obtained by any of the instruments control modules
   b. Inflow Pressure. Controls ventilation breathing pressure
   c. Inflow Time. Controls ventilation inhalation time
   d. Hold Time. Controls time inflow air/O₂ is maintained
   e. Outflow Pressure. Controls exhalation pressure to prevent atelectasis
   f. Outflow Time. Controls the exhalation time
   g. A-F allow the operator to set the parameters and the operation of the ventilator
   h. Graphics
      i. Inflow and outflow pressure are displayed with a bar graph
      ii. Inflow, hold and outflow time are displayed with a bar graph
      iii. Reservoir holding tank pressure is displayed digitally

It should be understood that the figures in the attachments, which highlight the structure, methodology, functionality and advantages of this disclosure, are presented for example purposes only. This disclosure is sufficiently flexible and configurable, such that it may be implemented in ways other than that shown in the accompanying figures.

What is claimed is:

1. A system for medical ventilation, the system comprising:
at least one medical sealed apparatus for storing pressurized gases, the pressurized gases comprising at least one of oxygen and air;
at least one breathing tube; and
a control mechanism
for coupling the at least one medical sealed apparatus with and the at least one breathing tube,
for blending the pressurized gases into a mix; and
for managing at least one inhalation cycle of the mixed pressurized gasses to the at least one breathing tube; and
for managing at least one exhalation cycle from the breathing tube, the at least one exhalation cycle generating an output.

2. The system of claim 1, wherein the control mechanism alters the mix of the pressurized gases in response to the output from the at least one exhalation cycle.

3. The system of claim 2, wherein the mix of the pressurized gases comprises a controlled mix of air and oxygen from 0% oxygen to 100% oxygen.

4. The system of claim 3, wherein the mix of pressurized gases comprises 5 pounds force per square inch to 20 pounds force per square inch.

5. The system of claim 4, wherein the control mechanism comprises:
a computer for managing an onset of the inhalation cycle in response to a volume of the output from the exhalation cycle; and
a timer for measuring at least one purge time between at least one pair of the one inhalation cycle and the exhalation cycle.

6. The system of claim 5, wherein the control mechanism further comprises a device for controlling a pressure of the at least one exhalation cycle and for allowing for carbon dioxide release.

7. The system of claim 6, wherein the control mechanism further comprises:
at least one closed-loop sensor for sensing pressure and flow of the mix,
wherein the closed-loop sensor controls the at least one inhalation cycle and the at least one exhalation cycle.

8. The system of claim 7, further comprising:
a monitor for monitoring the output to provide a constant and uniform purge of oxygen and air and for minimizing supply pressure fluctuation.

9. A system for medical ventilation for patients, the system comprising:
at least one medical sealed apparatus for storing pressurized gases, the pressurized gases comprising at least one of oxygen and air;
at least one breathing tube;
a control mechanism
for coupling the at least one medical sealed apparatus with the at least one breathing tube;
for blending the pressured oxygen and the pressurized air into a mix;
for managing at least one inhalation cycle of the mixed pressurized gases into the at least one breathing tube; and
for managing at least one exhalation cycle through the breathing tube, the at least one exhalation cycle generating an output, wherein the control mechanism further comprises:
a force gas flow device for generating a gas flow of at least one pressure gradient from the control mechanism; and
a monitor for monitoring the pressure gradient before the at least one exhalation cycle.

10. The system of claim 9, wherein the control mechanism alters the mix of the pressurized gases in response to the output from the at least one exhalation cycle.

11. The system of claim 10, wherein the mix of at least one pressurized gases comprises a controlled mix of air and oxygen from 0% oxygen to 100% oxygen.

12. The system of claim 11, wherein the mix of pressurized gases comprises 5 pounds force per square inch to 20 pounds force per square inch.

13. The system of claim 12, wherein the control mechanism comprises:
a computer for managing an onset of the inhalation cycle in response to a volume of the output from the exhalation cycle; and
a timer for measuring at least one purge time between at least one pair of the at least one inhalation cycle and the at least exhalation cycle.

14. The system of claim 13, wherein the control mechanism further controls a pressure of the at least one exhalation cycle to allow for carbon dioxide release.

15. The system of claim 14, wherein the monitor further comprises:
a monitoring controller for providing a constant uniform purge of oxygen and air, and for minimizing supply pressure fluctuation.

16. The system of claim 15, further comprising:
at least one closed-loop sensor for sensing pressure and flow of the mix, wherein the sensor controls the at least one inhalation and at least one exhalation cycle.

17. A method for ventilating medical patients, the method comprising:
storing at least one of pressurized oxygen and pressurized air in at least one medical sealed apparatus;
coupling the at least one medical sealed apparatus with at least one breathing tube;
blending the pressurized gases to a mix;
managing at least one inhalation cycle of the mix of pressurized gases into the at least breathing tube;
managing at least one exhalation cycle of through the at least one breathing tube;
generating an output through the at least one exhalation cycle;
generating a gas flow of at least one pressure gradient during the at least one exhalation cycle; and
monitoring the pressure gradient before the output is generated.

18. The method of claim 17, further comprising:
altering the mix of the pressurized oxygen and the pressurized air in response to the output during the at least one exhalation cycle.

19. The method of claim 18, wherein the mix of pressurized gases comprises a controlled mix of air and oxygen from 0% oxygen to 100% oxygen.

20. The method of claim 19, wherein the mix of pressurized gases comprises 5 pounds force per square inch to pounds force per square inch.

21. The method of claim 20, further comprising:
managing an onset of the inhalation cycle in response to a volume of the output from the exhalation cycle; and
measuring at least one purge time between at least one pair of the at least one inhalation cycle and the at least one exhalation cycle.

22. The method of claim 21, further comprising:
controlling an at least one exhalation cycle pressure to allow for carbon dioxide release.

23. The method of claim 22, further comprising:
providing a constant and uniform purge of oxygen and air while minimizing supply pressure fluctuation.

24. The method of claim 23, further comprising:
closed-loop sensing of pressure and flow of the mix to controls the at least one inhalation and at least one exhalation cycle.
